(19) United States
(12) Patent Application Publication
(10) Pub. No.: US 2019/0179883 A1
(43) Pub. Date: Jun. 13, 2019

(54) EVALUATING TEXTUAL ANNOTATION MODEL PERFORMANCE

(71) Applicant: INTERNATIONAL BUSINESS MACHINES CORPORATION, Armonk, NY (US)

(72) Inventors: Sheng Hua Bao, San Jose, CA (US); Robert Ip, San Jose, CA (US); Pathirage Perera, San Jose, CA (US); Cartic Ramakrishnan, San Jose, CA (US); Ramani Routray, San Jose, CA (US); Sundari Voruganti, San Jose, CA (US)

(21) Appl. No.: 15/836,250

(22) Filed: Dec. 8, 2017

(51) Int. Cl. G06F 17/24; G06F 17/27; G16H 10/60; G06F 15/18

(52) U.S. Cl. CPC G06F 17/241; G06F 15/18; G16H 10/60; G06F 17/277

(57) ABSTRACT

Evaluation of textual annotation models is provided. In various embodiments, an annotation model is applied to textual training data to derive a plurality of automatic annotations. The plurality of automatic annotations is compared to ground truth annotations of the textual data to determine overlapping tokens between the plurality of automatic annotations and the ground truth annotations. Weights are assigned to the overlapping tokens. Based on the weights of the overlapping tokens, scores are determined for the automatic annotations. The scores indicate the correctness of the automatic annotations relative to the ground truth annotations. Based on the scores of for the automatic annotations, an accuracy of the annotation model is determined.

### BACKGROUND

Embodiments of the present disclosure relate to medical annotation, and more specifically, to evaluating textual annotation model performance.

### BRIEF SUMMARY

According to embodiments of the present disclosure, methods of and computer program products for evaluating an annotation model are provided. In various embodiments, an annotation model is applied to textual training data to derive a plurality of automatic annotations. The plurality of automatic annotations is compared to ground truth annotations of the textual data to determine overlapping tokens between the plurality of automatic annotations and the ground truth annotations. Weights are assigned to the overlapping tokens. Based on the weights of the overlapping tokens, scores are determined for the automatic annotations. The scores indicate the correctness of the automatic annotations relative to the ground truth annotations. Based on the scores of for the automatic annotations, an accuracy of the annotation model is determined.

### BRIEF DESCRIPTION OF THE SEVERAL VIEWS OF THE DRAWINGS

FIG. 1 illustrates a method of evaluating an annotation model according to embodiments of the present disclosure.

FIG. 2 illustrates an example annotation and associated vocabulary weights according to embodiments of the present disclosure.

FIG. 3 illustrates a method of training the evaluation of an annotation model according to embodiments of the present disclosure.

FIG. 4, illustrates a method of evaluating an annotation model according to embodiments of the present disclosure.

FIG. 5 depicts a computing node according to an embodiment of the present invention.

### DETAILED DESCRIPTION

Various natural language processing tasks rely on entity extraction, segmentation, and annotation. Particularly in knowledge extraction from unstructured textual data, entities of importance must be identified and annotated accurately. Likewise, automatic annotation of medical records requires accurate text segmentation.

Various methods are available for automatic annotation of medical records, including application of predetermined extraction rules such as regular expressions, as well as learning systems. Learning systems are generally trained based on expert annotated training data. A variety of learning systems are known in the art, including linear classifiers, support vector machines (SVMs), and artificial neural networks (ANNs) such as recurrent neural networks (RNNs). Various systems are also available for automatically generating human readable annotation rules such as regular expressions.

To train a learning system, a pre-annotated corpus of medical records is provided. Based on the ground truth, which generally include expert annotations, the learning system is trained to reach a similar annotation result as an expert. For example, when the learning system comprises an artificial neural network, backpropagation is one suitable algorithm for supervised learning, in which a known correct output is available during the learning process. The goal of such learning is to obtain a system that generalizes to data that were not available during training.

During backpropagation, the output of the artificial neural network is compared to the known correct output for each input example. An error value is calculated for each of the neurons in the output layer. The error values are then used to update the weights of the artificial neural network. By incremental correction in this way, the network output is adjusted to conform to the training data. When applying backpropagation, an ANN rapidly attains a high accuracy on most of the examples in a training-set.

During the training process, the known correct output (or ground truth) is compared to the learning system's output. Likewise, during evaluation and comparison of multiple automatic annotation approaches, the ground truth is compared to the automatic annotation. The correctness of an annotation is generally measured in terms of an exact match between the output of a machine learning model and the ground truth. This correctness may be used to determine an overall accuracy score for a given model. For example, overall accuracy may be determined according to precision, recall, F₁ score, or Cohen's kappa, among other available metrics.

However, such a strict requirement for correctness of the output does not necessarily provide the most accurate picture of overall model performance. In particular, inclusion or omission of certain words may have a different degree of importance to the overall annotation. Thus, a strict comparison between output and ground truth may misjudge the overall accuracy of a model that, for example, only omits unimportant words as compared to the ground truth.

Accordingly, the present disclosure provides for fuzzy matching between annotation and ground truth to more accurately measure correctness of a result, and thereby improve the evaluation of annotation models. The fuzzy matching provided herein may also be used to identify and evaluate discrepancies in ground truth.

In various embodiments, keywords are identified that are indicative of important events, for example a medically adverse event. The identified keywords are assigned weights indicative of their importance to annotation. These weights are used to assign a probability of correctness metric. It will be appreciated that although various examples described herein relate to annotation, this correctness measure may be applied to a variety of segmentation, entity extraction, or other natural language processing processes. It will also be appreciated that although various examples provided herein refer to words, the present disclosure is applicable to any token of an input string, whether a word or other character string such as a number or date.

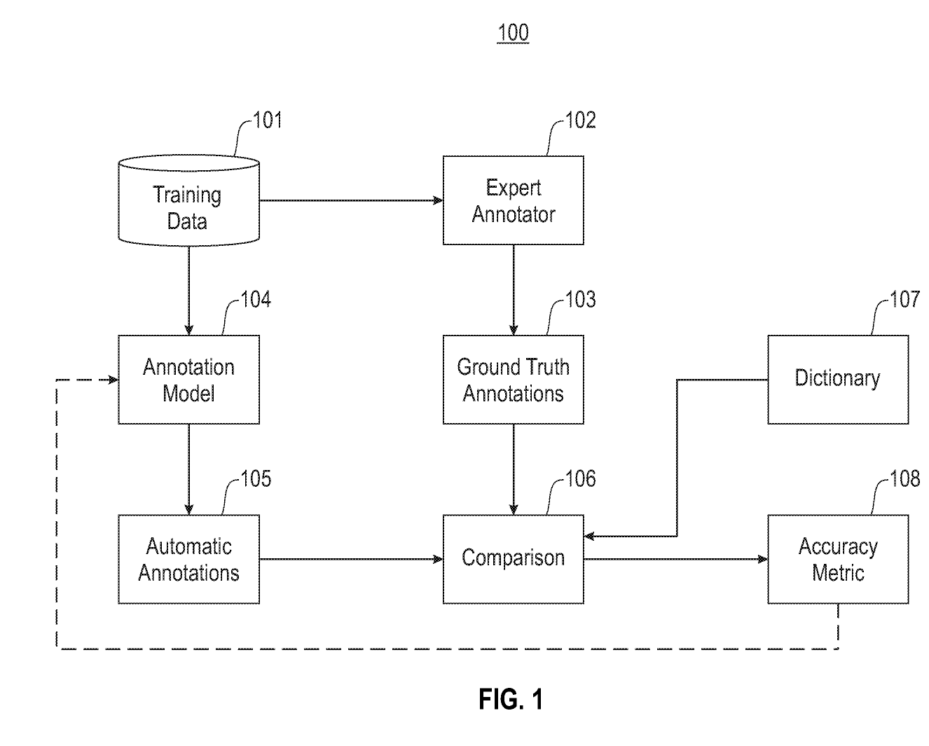

With reference now to FIG. 1, a method for evaluating annotation model performance is illustrated according to embodiments of the present disclosure. Training data 101 are provided to expert annotator 102. In various embodiments, training data comprise electronic health records. In general, an electronic health record (EHR), or electronic medical record (EMR), may refer to the systematized collection of patient and population electronically-stored health information in a digital format. These records can be shared across different health care settings and may extend beyond the information available in a PACS. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

The expert annotator 102 may be instructed to perform a variety of annotation tasks. For example, they may be instructed to highlight adverse events in the body of input text. Exemplary text for annotation is included in Inset 1 below, with adverse events underlined.

Inset 1

Participant JB (0005) presented to the emergency room on
Aug. 13, 2016. He reported nausea, vomiting, and diarrhea
for several days and had an 18 lb weight decrease since
Jul. 31, 2016.
Robin, Pharmacist, stated patient's white blood count was
low and his Lenalidomide does is being adjusted.

From the above, it will be appreciated that adverse event indicators in text may span more than one word or other token. In some cases, adverse events may have various alternative wordings that represent the same adverse event, as illustrated in Table 1. Moreover, some words within a span of words may be more important than others, relatively, when identifying adverse events. For example, in the phrase “his liver numbers were not doing well”, the word “not” is of critical importance as it differentiates a positive meaning from a negative meaning.

TABLE 1

| First Phrasing | Second Phrasing |
|---|---|
| swelling around ankles | swelling of ankles |
| feet swelling | swelling of the feet |
| swelling in her body | swelling from head to toe |
| patient not tolerating well | not tolerating it well |
| not feeling well | not been doing well |
| patient not doing well with current dose | not working well enough |

An expert annotator is able to identify an adverse event irrespective of wording, and arrive at an accurate annotation. In this way, ground truth annotations 103 are determined.

To evaluate annotation model 104, the annotation model is also provided with training data 101. As noted above, the annotation model may include one of various learning systems. Annotation model 104 outputs automatic annotations 105 for training data 101. A high quality annotation model will generally arrive at the same or nearly the same annotations as the expect annotator.

To determine the accuracy of annotation model 104, each of automatic annotations 105 is compared with corresponding ground truth annotations 103. In some embodiments, comparison 106 leverages dictionary 107, which contains a subject matter specific vocabulary accompanied by weights for each entry. As described above, in some embodiments, dictionary 107 associates weights with keywords based on importance within a healthcare context. In some embodiments, the weighting is based on frequency within the medical context.

In some embodiments, comparison 106 determines for each pair of automatic annotation and ground truth annotation, the number of words or other tokens overlapping for each annotation. Thus, in a five word phrase, a score of 100% would indicate a complete match, while a score of 80% would indicate one extra word included or omitted.

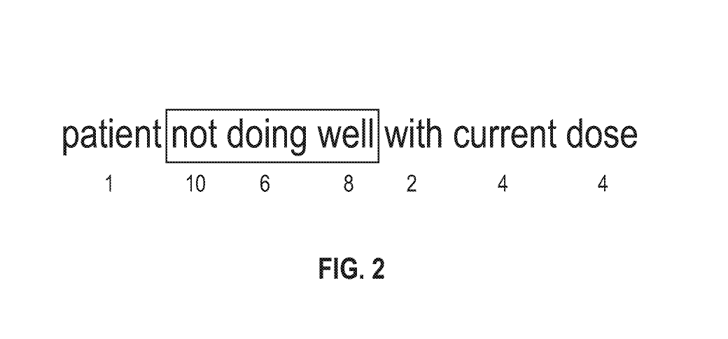

In some embodiments, comparison 106 determines for each pair of automatic annotation and ground truth annotation, the relative weight of words or other tokens overlapping for each annotation. Referring to FIG. 2, an example annotation and vocabulary weights are provided. In this example, the exemplary phrase “patient not doing well with current dose” was included in ground truth annotations 103. The phrase “not doing well” was automatically annotated. The weights of each individual word are given below. Given these weights, the total concurrence between the ground truth and the automatic annotations is 10+6+8=24. The total of the non-overlapping weights is 1+2+4+4=11. Thus, the weighted overlap between the ground truth and the output of annotation model 104 for this example is 24/36=66.7%.

Based on the above scaled correctness measurement for each annotation, and overall accuracy of annotation model 104 may be computed. As noted above, precision, recall, F₁ score, or Cohen's kappa may be used to evaluate the performance of an annotation model. To adopt a simple example for illustrative purposes, the average correctness measurement may simply be taken across all samples in the training data. In this example, while a exact match requirement may result in a relatively low overall accuracy measure of the model, the scaled correctness measurement may provide a higher and more useful accuracy measure due to fuzzy matching.

As noted above, the accuracy metric may be used for further training of annotation model 104, for example by providing an indication of correctness or incorrectness. In some embodiments, a fixed threshold may be adopted for correctness when training the model. However, it will be appreciated that certain models may be trained based on a relative indication of correctness such as the scaled value created in comparison 106.

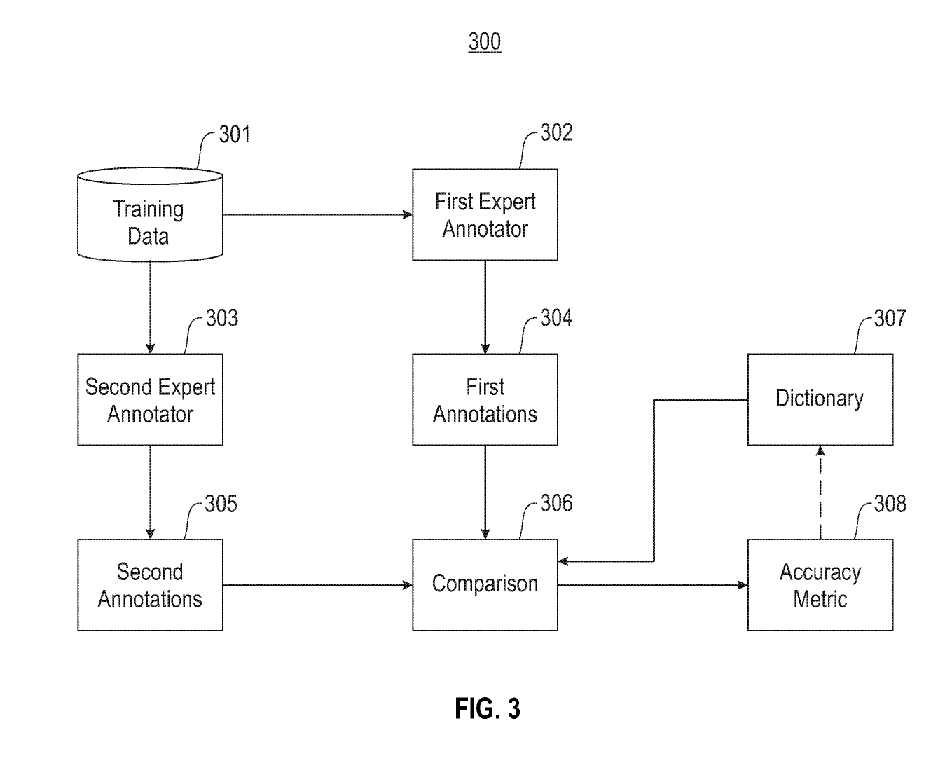

Referring now to FIG. 3, a method of training the evaluation of an annotation model is illustrated according to embodiments of the present disclosure. Training data 301 are provided to both first expert annotator 302 and second expert annotator 303, who provide first annotations 304 and second annotations 305. A comparison 306 is made between each corresponding pair of first annotations 304 and second annotations 305. Different subject matter experts may label the same data differently. Words or other tokens that overlap are provided higher weights in dictionary 307. Words or other tokens that do not overlap are given lower weights in dictionary 307. In this way, the disparity in expert annotation allows an inference of the important of each word or other token in the dictionary to an accurate annotation. In some embodiments, the weight may be a simple count of overlapping occurrences, which may be normalized by the total number of terms considered. However, it will be appreciated that a variety of techniques may be applied to increment or decrement weights in dictionary 307.

In some embodiments, a an expert reviewer is used to train the appropriate weights for each dictionary term. In such embodiments, a user may be presented with automatic annotations 105 and ground truth annotations 103. The user may indicate which non-overlapping words or other tokens are in fact important, thereby increasing their weights.

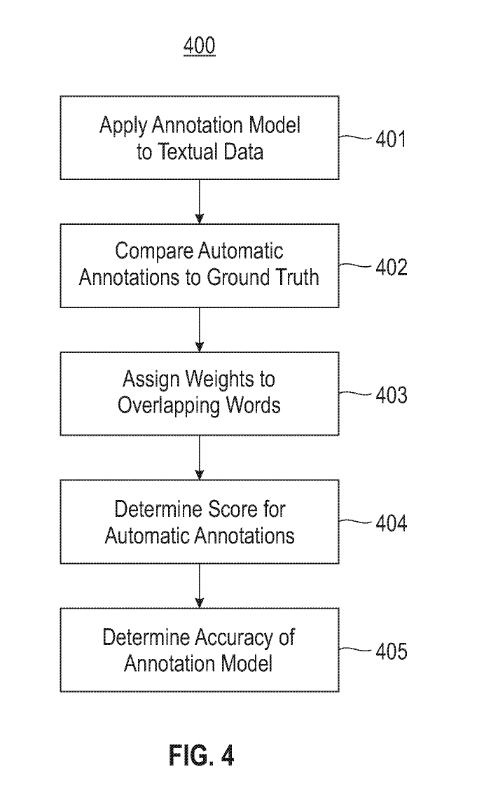

Referring now to FIG. 4, a method for evaluating an annotation model is illustrated according to the present disclosure. At 401, an annotation model is applied to textual training data to derive a plurality of automatic annotations. At 402, the plurality of automatic annotations is compared to ground truth annotations of the textual data to determine overlapping tokens between the plurality of automatic annotations and the ground truth annotations. At 403, weights are assigned to the overlapping tokens. At 404, based on the weights of the overlapping tokens, scores are determined for the automatic annotations. The scores indicate the correctness of the automatic annotations relative to the ground truth annotations. At 405, based on the scores of for the automatic annotations, an accuracy of the annotation model is determined.

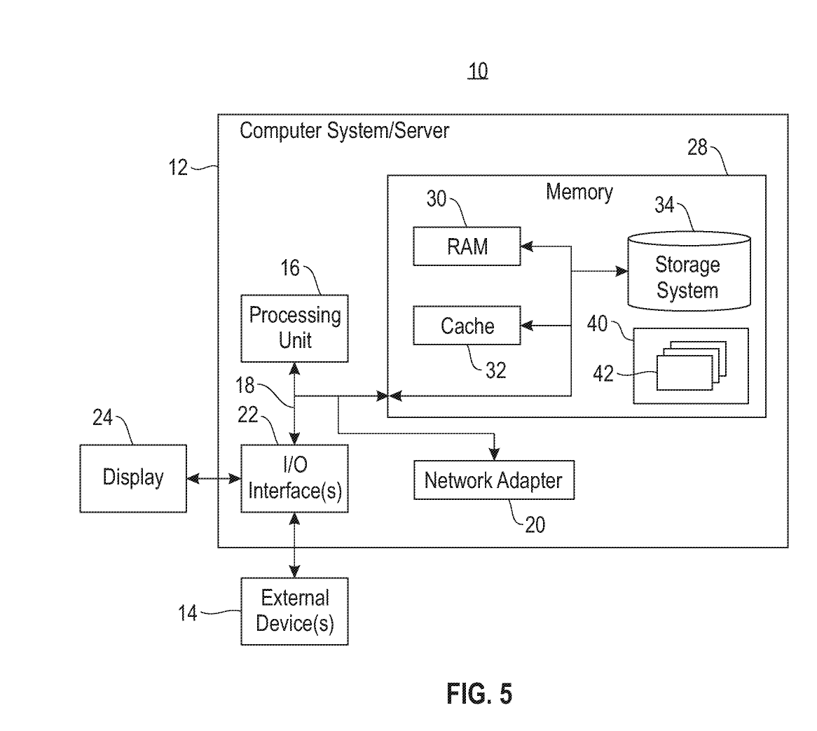

Referring now to FIG. 5, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
applying an annotation model to textual training data to derive a plurality of automatic annotations;
comparing the plurality of automatic annotations to ground truth annotations of the textual data to determine overlapping tokens between the plurality of automatic annotations and the ground truth annotations;
assigning weights to the overlapping tokens;
based on the weights of the overlapping tokens, determining scores for the automatic annotations, the scores indicating the correctness of the automatic annotations relative to the ground truth annotations for determining an accuracy of the annotation model.

2. The method of claim 1, wherein the textual training data comprise medical records.

3. The method of claim 1, wherein assigning weights to the overlapping tokens comprises retrieving weights from a dictionary of terms.

4. The method of claim 1, wherein the weights correspond to the frequency of the overlapping tokens in a corpus.

5. The method of claim 1, wherein the weights correspond to the important of the overlapping tokens within a corpus.

6. The method of claim 1, wherein determining the scores for the automatic annotations comprises determining a ratio of the weights of the overlapping tokens to the weights of all tokens in the ground truth annotations.

7. The method of claim 1, wherein determining the accuracy of the annotation model comprises averaging the scores for the automatic annotations.

8. The method of claim 1, wherein the annotation model identifies adverse events in the textual data.

9. A system comprising:
a computing node comprising a computer readable storage medium having program instructions embodied therewith, the program instructions executable by a processor of the computing node to cause the processor to perform a method comprising:
applying an annotation model to textual training data to derive a plurality of automatic annotations;
comparing the plurality of automatic annotations to ground truth annotations of the textual data to determine overlapping tokens between the plurality of automatic annotations and the ground truth annotations;
assigning weights to the overlapping tokens;
based on the weights of the overlapping tokens, determining scores for the automatic annotations, the scores indicating the correctness of the automatic annotations relative to the ground truth annotations for determining an accuracy of the annotation model.

10. The system of claim 9, wherein the textual training data comprise medical records.

11. The system of claim 9, wherein assigning weights to the overlapping tokens comprises retrieving weights from a dictionary of terms.

12. The system of claim 9, wherein the weights correspond to the frequency of the overlapping tokens in a corpus.

13. A computer program product for evaluating an annotation model, the computer program product comprising a computer readable storage medium having program instructions embodied therewith, the program instructions executable by a processor to cause the processor to perform a method comprising:
applying an annotation model to textual training data to derive a plurality of automatic annotations;
comparing the plurality of automatic annotations to ground truth annotations of the textual data to determine overlapping tokens between the plurality of automatic annotations and the ground truth annotations;
assigning weights to the overlapping tokens;
based on the weights of the overlapping tokens, determining scores for the automatic annotations, the scores indicating the correctness of the automatic annotations relative to the ground truth annotations for determining an accuracy of the annotation model.

14. The computer program product of claim 13, wherein the textual training data comprise medical records.

15. The computer program product of claim 13, wherein assigning weights to the overlapping tokens comprises retrieving weights from a dictionary of terms.

16. The computer program product of claim 13, wherein the weights correspond to the frequency of the overlapping tokens in a corpus.

17. The computer program product of claim 13, wherein the weights correspond to the important of the overlapping tokens within a corpus.

18. The computer program product of claim 13, wherein determining the scores for the automatic annotations comprises determining a ratio of the weights of the overlapping tokens to the weights of all tokens in the ground truth annotations.

19. The computer program product of claim 13, wherein determining the accuracy of the annotation model comprises averaging the scores for the automatic annotations.

20. The computer program product of claim 13, wherein the annotation model identifies adverse events in the textual data.
